(19) United States
(12) Patent Application Publication
(10) Pub. No.: US 2025/0368707 A1
(43) Pub. Date: Dec. 4, 2025

(54) BIFUNCTIONAL IL-2 AND IL-10 FUSION PROTEINS AND USES THEREOF

(71) Applicant: UNIVERSITY OF MIAMI, Miami, FL (US)

(72) Inventors: Thomas R. MALEK, Miami, FL (US); Alicia Santos SAVIO, Miami, FL (US)

(21) Appl. No.: 18/870,189

(22) PCT Filed: Jun. 1, 2023
(86) PCT No.: PCT/US2023/067748

§ 371 Date: Nov. 27, 2024

Related U.S. Application Data
(60) Provisional application No. 63/347,684, filed on Jun. 1, 2022.

(51) Int. Cl. C07K 14/55; A61K 38/00; A61P 3/10; A61P 29/00; A61P 37/06; C07K 14/54; C07K 14/715

(52) U.S. Cl. CPC C07K 14/55; A61P 3/10; A61P 29/00; A61P 37/06; C07K 14/5428; C07K 14/7155; A61K 38/00; C07K 2319/00

(57) ABSTRACT

The present disclosure relates to recombinant polypeptides and uses thereof for treating, preventing, and detecting inflammatory diseases. Specifically, the disclosure provides a recombinant polypeptide comprising an IL-2 polypeptide, a CD25 polypeptide, and an IL-10 polypeptide, wherein the CD25 polypeptide comprises an extracellular domain of a CD25 protein. In some embodiments, the IL-10 polypeptide is linked to the C-terminus of the CD25 polypeptide.

### CROSS REFERENCE TO RELATED APPLICATIONS

This application claims the benefit of U.S. Provisional patent application Ser. No. 63/347,684 filed Jun. 1, 2022, the disclosure of which is expressly incorporated herein by reference in its entirety.

### STATEMENT REGARDING FEDERALLY SPONSORED RESEARCH

This invention was made with government support under Grant No. R21AI159489 awarded by the National Institute of Allergy and Infectious Diseases. The government has certain rights in the invention.

### FIELD

The present disclosure relates to recombinant polypeptides and uses thereof for treating, preventing, and detecting inflammatory diseases.

### BACKGROUND

At least 80 distinct autoimmune diseases have been recognized by the CDC that affect approximately 20% of the population in the USA, with more women being affected than men. The cost to the health care system was estimated to be approximately $100 billion/year in a report released about 10 years ago by the American Autoimmune Related Diseases Association. Current therapies are inadequate as they treat symptoms, not the underlying cause, and are often associated with severe side effects, especially upon prolonged use. Regulatory T cells (Tregs) represent a major mechanism to actively suppress self-reactive T cells, which are present in all individuals. A breakdown in Tregs leads to activation of self-reactive T cells that contribute to the development of autoimmunity. Correspondingly, an attractive therapeutic approach is to re-regulate the immune system to boost the numbers and/or function of Tregs to suppress autoreactive T cells. What is needed are novel compositions and methods for treating autoimmune diseases through the regulation of Tregs.

### SUMMARY

The present disclosure relates to recombinant polypeptides and uses thereof for treating autoimmune diseases.

Accordingly, in some aspects, disclosed herein is a recombinant polypeptide comprising:
an IL-2 polypeptide;
a CD25 polypeptide; and
an IL-10 polypeptide.

In some embodiments, wherein the CD25 polypeptide comprises an extracellular domain of a CD25 protein. In some embodiments, the IL-10 polypeptide is linked to the C-terminus of the CD25 polypeptide.

In some embodiments, the IL-2 polypeptide comprises a sequence at least 80% identical to SEQ ID NO: 1 or 7 or a fragment thereof. In some embodiments, the IL-2 polypeptide comprises a C145S mutation relative to SEQ ID NO: 7 or a fragment thereof. In some embodiments, the IL-2 polypeptide comprises the sequence of SEQ ID NO: 4 or a fragment thereof.

In some embodiments, the CD25 polypeptide comprises a truncated C-terminus. In some embodiments, the CD25 polypeptide comprises a truncated C-terminus from residues 213 to 240 of the extracytoplasmic domain residues of CD25.

In some embodiments, the CD25 polypeptide comprises a sequence at least 80% identical to SEQ ID NO: 2 or 5 or a fragment thereof. In some embodiments, the IL-10 polypeptide comprises a sequence at least 80% identical to SEQ ID NO: 3 or 6 or a fragment thereof.

In some embodiments, the recombinant polypeptide of any preceding aspect comprises a sequence at least 80% identical to SEQ ID NO: 8 or 9 or a fragment thereof.

In some aspects, disclosed herein is a recombinant polynucleotide comprising a nucleic acid sequence encoding the recombinant polypeptide of any preceding aspect. In some embodiments, the nucleic acid sequence is at least 80% identical to SEQ ID NO: 10 or 17 or a fragment thereof.

In some aspects, disclosed herein is a vector comprising the recombinant polynucleotide of any preceding aspect.

In some aspects, disclosed herein is a method of treating an inflammatory disease in a subject in need thereof, comprising administering to the subject a therapeutically effective amount of the recombinant polypeptide or the recombinant polynucleotide of any preceding aspect. In some embodiments, the inflammatory disease comprises systemic lupus erythematosus (SLE), multiple sclerosis, Addison disease, graft-versus-host disease, transplant rejection reactions, asthma, type 1 diabetes (T1D), alopecia areata, rheumatoid arthritis, ankylosing spondylitis, psoriasis, Behcet's disease, granulomatosis with polyangiitis, Takayasu's disease, Crohn's disease, ulcerative colitis, Grave's disease, Hashimoto thyroiditis, myasthenia gravis, Sjogren syndrome, Celiac disease, pernicious anemia, psoriatic arthritis, autoimmune hepatitis, sclerosing cholangitis, Bullous pemphigoid, Juvenile idiopathic arthritis, scleroderma, hemolytic anemia, systemic sclerosis, Pemphigas, Gougerot-sjogrens, macrophage activating syndrome, Alzheimer's disease, myocarditis, or sepsis. In some embodiments, the inflammatory disease is type 1 diabetes (TID).

### DESCRIPTION OF DRAWINGS

The accompanying figures, which are incorporated in and constitute a part of this specification, illustrate aspects described below.

FIG. 1 shows model of mIL-2/CD25.

FIG. 2 shows that mIL-2/CD25 selectively activates Tregs. Dose-response curves of in vitro STA T5 activation (mean+/−SEM) of the indicated cell populations in the spleen after incubation with IL-2 (n=3) or mIL-2/CD25 (n=2) for 15 min.

FIGS. 3A-3B shows that mIL-2/CD25 limits diabetes in female NOD mice. (FIG. 3A) shows 6-7 week-old (n=8-10/group) or (FIG. 3B) shows hyperglycemic (glucose 150-250 mg/dl, n=26 mice/group) female NOD mice that received the indicated dose of mIL-2/CD25, human IL-2, or HSS twice a week for 5-10 weeks (blue bar). Diabetes free-survival were analyzed by Mantel-Cox log-rank test.

FIG. 4 shows model of mIL-2-10/CD25.

FIGS. 5A-5B show biochemical characterization of nickel affinity purified mIL-2-10/CD25. (FIG. 5A) SDS-PAGE and (FIG. 5B) size exclusion chromatography with multiple light scatter (SEC-MALS) under native conditions using PBS. The molecular mass is shown for the major peak.

FIG. 6 shows the IL-2 activity of mIL-2-10/CD25 when assessed in comparison to mouse IL-2 and mIL-2/CD25 by monitoring the growth of CTLL cells using MTT.

FIG. 7 shows IL-10 activity of mIL-2-10/CD25 when determined in comparison to mouse IL-10 by activation of STAT3 after stimulation of RAW 264.7 cells for 15 min in vitro.

FIGS. 8A-8B show the IL-10 activity of mIL-2-10/CD25 when determined by inhibition of secretion of IFNγ or IL-6 after stimulation of C57BL/6 spleen cells (n=4) with anti-CD3 (FIG. 8A) or LPS (FIG. 8B) for 24 hr in the presence of mIL-2 (1 ng/ml), mIL-10 (10 ng/ml), mIL-2/CD25 (1 μg/ml), or mIL-2-10/CD25 (1 μg/ml). Data (mean±SEM) were analyzed by a one-way ANOVA. ***p<0.001; ****p<0.0001.

FIG. 9 shows that mIL-2-10/CD25 has bifunctional activity in vivo. Assessment of IL-2-dependent activation of pSTAT5 and IL-10-dependent activation of pSTAT3. C57BL/6 mice (n=3) received a single injection of PBS, mIL-2/CD25 (5 μg), mIL-2-10/CD25 (50 μg), mIL-2 (2 μg), or mIL-10 (2 μg). 90 min later, pSTAT5 and pSTAT3 were assessed for Tregs and CD11b+ macrophages directly ex vivo. Data (mean±SEM) were analyzed by one-way ANOVA using Tukey's multiple comparison test. *p<0.05; **P<0.01; ****p<0.0001.

FIG. 10 shows that mIL-2-10/CD25 has bifunctional activity in vivo. Assessment of IL-2 activity by expansion of Tregs. C57BL/6 mice (n=3) received a single injection of mIL-2/CD25 (5 μg), mIL-2-10/CD25 (50 μg), or daily injections of PBS, mIL-2 (2 μg), or mIL-10 (2 μg). Expansion of splenic Tregs and their proliferation (Ki67+) was assessed 72 hr later. Data (mean±SEM) were analyzed by one-way ANOVA using Tukey's multiple comparison test. *p<0.05; ****p<0.0001.

FIG. 11 shows SDS-PAGE under reducing conditions of purified hIL-2-10/tCD25 identified by Coomassie Blue staining (left) or Western blotting (right) using anti-IL-10.

FIG. 12 shows molecular characteristics of native hIL-2-10/tCD25. Elution profile of size exclusion chromatography (SEC) using Sephacryl S300 of nickel column affinity purified hIL-2-10/tCD25 (top). Identification of dimers of hIL-2-10/CD25 by non-denaturing PAGE of individual fractions from the S300 column (bottom). The major peak in A (fraction 26-35) are largely dimers (B).

FIG. 13 shows the IL-2 activity of hIL-2-10/tCD25 in comparison to hIL-2/CD25 and hIL-2 when assessed by monitoring the growth of CTLL cells using MTT. Medium refers to cultures of CTLL without addition of cytokines or fusion proteins.

FIG. 14 shows IL-10 activity of hIL-2-10/tCD25 when determined in comparison to human and mouse IL-10 by activation of STAT3 after stimulation of RAW 264.7 cells for 15 min in vitro.

FIG. 15 shows that hIL-2-10/CD25 has bifunctional activity in vivo. IL-2 activity was assessed by increased Tregs in the spleen. C57BL/6 mice received a single injection of PBS, hIL-2/CD25 (100 μg), or hIL-2-10/CD25 (150 μg) 72 hr later Tregs were quantified based on Foxp3+ cells within total gated CD4+ T cells.

FIG. 16 shows that hIL-2-10/CD25 has bifunctional activity in vivo. Assessment of IL-10 activity. C57BL/6 mice (n=5) received 100 μg of LPS. 30 min later the mice received PBS, mIL-2/CD25 (5 μg), mIL-2-10/CD25 (50 μg), hIL-2-10/tCD25 (50 μg), or mIL-10 (2 μg). Hyperthermia was assessed for 24 hr. Data are represented as the mean±SEM.

FIG. 17 shows that hIL-2-10/CD25 has bifunctional activity in vivo. Assessment of IL-10 activity. C57BL/6 mice (n=5) received 100 μg of LPS. 30 min later the mice received PBS, mIL-2/CD25 (5 μg), mIL-2-10/CD25 (50 μg), hIL-2-10/tCD25 (50 μg), or mIL-10 (2 μg). Mice were bled 3 hr later and serum IL-6 and TNFα levels were determined by ELISA. Data (mean±SEM) were analyzed by one-way ANOVA using Tukey's multiple comparison test. ***p<0.001; ****p<0.0001.

FIG. 18 shows that mIL2-10/CD25 is highly effective in controlling autoimmunity. 12-week old female NOD mice (n=7-10) received PBS, Mil-2/CD25 (5 μg), or mIL-2-10/CD25 (50 μg) twice a week for 5 weeks by i.p. injection. Blood glucose was monitored twice per week, where mice were considered diabetic when blood glucose reached>400 mg/dl. Mice with acute diabetes were excluded from the analysis, i.e., diabetic at or before 15 weeks of age. Data were evaluated by Kaplan-Meier survival analysis.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### TERMINOLOGY

The term “about” as used herein when referring to a measurable value such as an amount, a percentage, and the like, is meant to encompass variations of +20%, +10%, +5%, or +1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a cancer or an inflammatory disease). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises the polypeptides or the polynucleotides dislcosed herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “control” is an alternative subject or sample used in an experiment for comparison purposes. A control can be “positive” or “negative.”

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant polypeptide” refers to an amount of a recombinant peptide sufficient to prevent, treat, or mitigate an inflammatory disease.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term as used herein “engineered” and other grammatical forms thereof may refer to one or more changes of nucleic acids, such as nucleic acids within the genome of an organism. The term “engineered” may refer to a change, addition and/or deletion of a gene. Engineered cells can also refer to cells that contain added, deleted, and/or changed genes.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is a hematologic cancer. In some embodiments, the cancer is caused by a solid tumor.

The term “primary tumor” refers to a tumor growing at the site of the cancer origin.

The term “metastatic tumor” refers to a secondary tumor growing at the site different from the site of the cancer origin.

A “recurrence” means that the cancer has returned after initial treatment.

“Non-recurrent” or “recurrence-free”, as used herein means that the cancer is in remission; being recurrent means that the cancer is growing and/or has metastasized, and some surgery, therapeutic intervention, and/or cancer treatment is required to lower the chance of lethality. The “non-recurrent subjects” are subjects who have non-recurrent or recurrence-free disease, and they can be used as the control for recurrent subjects who have recurrent disease or recurrence.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g., promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Therapeutically effective amount” refers to the amount of a composition that will elicit the biological or medical response of a tissue, system, animal, or human that is being sought by the researcher, veterinarian, medical doctor or other clinician over a generalized period of time. In some embodiments, a desired response is reduction of inflammation in a subject. In some embodiments, the desired response is prevention, treatment, and/or mitigation of an inflammatory disease and/or related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. It will be understood that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of an inflammatory disease or condition and/or alleviating, mitigating or impeding one or more causes of an inflammatory disease. Treatments according to the invention may be applied preventively, prophylactically, palliatively or remedially.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

Disclosed herein are the components to be used to prepare the disclosed compositions as to be used in the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### Recombinant Polypeptides and Polynucleotides

In some aspects, disclosed herein is a recombinant polypeptide comprising:
an IL-2 polypeptide;
a CD25 polypeptide; and
an IL-10 polypeptide.

In some embodiments, the CD25 polypeptide comprises an extracellular domain of a CD25 protein. In some embodiments, the IL-10 polypeptide is linked to the C-terminus of the CD25 polypeptide.

In some embodiments, the IL-2 polypeptide comprises a sequence at least 80% (for example, at least about 80%, about 85%, about 90%, about 95%, or about 98%) identical to SEQ ID NO: 1 or 7 or a fragment thereof. In some embodiments, the IL-2 polypeptide comprises a C145S mutation relative to SEQ ID NO: 7. In some embodiments, the IL-2 polypeptide comprises the sequence of SEQ ID NO: 4 or a fragment thereof. The recombinant IL-2-10/CD25 disclosed herein can reduce protein aggregation.

In some embodiments, the CD25 polypeptide comprises a truncated C-terminus. In some embodiments, the CD25 polypeptide comprises a truncated C-terminus from residues 213 to 240 of the extracytoplasmic domain residues of CD25. In some embodiments, the C-terminus truncation refers to amino acid residues 213 to 240 relative to a wild type CD25. In some embodiments, the wild type CD25 polypeptide comprises the sequence set forth in SEQ ID NO: 22.

In some embodiments, the CD25 polypeptide comprises a sequence at least 80% (for example, at least about 80%, about 85%, about 90%, about 95%, or about 98%) identical to SEQ ID NO: 2 or 5 or a fragment thereof.

In some embodiments, the IL-10 polypeptide comprises a sequence at least 80% (for example, at least about 80%, about 85%, about 90%, about 95%, or about 98%) identical to SEQ ID NO: 3 or 6 or a fragment thereof.

In some embodiments, the recombinant polypeptide of any preceding aspect comprises a sequence at least 80% (for example, at least about 80%, about 85%, about 90%, about 95%, or about 98%) identical to SEQ ID NO: 8 or a fragment thereof.

In some embodiments, the recombinant polypeptide of any preceding aspect comprises a sequence at least 80% (for example, at least about 80%, about 85%, about 90%, about 95%, or about 98%) identical to SEQ ID NO: 9 or a fragment thereof.

In some aspects, disclosed herein is a recombinant polynucleotide comprising a nucleic acid sequence encoding the recombinant polypeptide of any preceding aspect. In some embodiments, the nucleic acid sequence is at least 80% (for example, at least about 80%, about 85%, about 90%, about 95%, or about 98%) identical to SEQ ID NO: 10 or a fragment thereof.

In some aspects, disclosed herein is a recombinant polynucleotide comprising a nucleic acid sequence encoding the recombinant polypeptide of any preceding aspect. In some embodiments, the nucleic acid sequence is at least 80% (for example, at least about 80%, about 85%, about 90%, about 95%, or about 98%) identical to SEQ ID NO: 17 or a fragment thereof.

In some embodiments, the polypeptides of any preceding aspect are human polypeptides or murine polypeptides.

In some aspects, disclosed herein is a vector comprising the recombinant polynucleotide disclosed herein. Accordingly, in some aspects, disclosed herein is a recombinant polynucleotide encoding a recombinant polypeptide comprising:
an IL-2 polypeptide;
a CD25 polypeptide; and
an IL-10 polypeptide.

In some embodiments, the recombinant polynucleotide is at least 80% (for example, at least about 80%, about 85%, about 90%, about 95%, or about 98%) identical to SEQ ID NO: 10 or 17 or a fragment thereof. In some embodiments, the vector comprises an IL-12 polynucleotide at least 80% (for example, at least about 80%, about 85%, about 90%, about 95%, or about 98%) identical to SEQ ID NO: 12 or 19 or a fragment thereof. In some embodiments, the vector comprises a CD25 polynucleotide at least 80% (for example, at least about 80%, about 85%, about 90%, about 95%, or about 98%) identical to SEQ ID NO: 15 or 20 or a fragment thereof. In some embodiments, the vector comprises an IL-10 polynucleotide at least 80% (for example, at least about 80%, about 85%, about 90%, about 95%, or about 98%) identical to SEQ ID NO: 16 or 21 or a fragment thereof. In some embodiments, the recombinant polypeptide further comprises one or more linker sequences, wherein the linker sequence comprises a sequence at least 80% (for example, at least about 80%, about 85%, about 90%, about 95%, or about 98%) identical to SEQ ID NO: 13 or 14 or a fragment thereof. In some embodiments, the recombinant polypeptide further comprises a signal sequence, wherein the signal sequence comprises a sequence at least 80% (for example, at least about 80%, about 85%, about 90%, about 95%, or about 98%) identical to SEQ ID NO: 11 or 18 or a fragment thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell. The term “viral vector” is also meant to refer to those forms described more fully in U.S. Patent Application Publication U.S. 2018/0057839, which is incorporated herein by reference for all purposes. Suitable viral vectors include, e.g., adenoviruses, adeno-associated virus (AAV), vaccinia viruses, herpesviruses, baculoviruses and retroviruses, parvoviruses, and lentiviruses. In some embodiments, the viral vector is a lentiviral vector. Methods for gene delivery are known in the art. See, e.g., U.S. Pat. Nos. 5,399,346, 5,580,859, 5,589,466, incorporated by reference herein in their entireties.

In certain embodiments, the disclosure contemplates pharmaceutical compositions comprising polypeptides disclosed herein, or optionally other pharmaceutical agent, or pharmaceutically acceptable salts thereof, and a pharmaceutically acceptable excipient.

In certain embodiments, this disclosure relates to compositions such as pharmaceutical compositions and cell growth media comprising peptides disclosed herein. In certain embodiments, this disclosure relates to pharmaceutical compositions comprising any recombinant polypeptides disclosed herein (such as, for example, SEQ ID NO: 8 or 9 or a fragment thereof) and a pharmaceutically acceptable excipient. In certain embodiments, the pharmaceutical composition is in the form of a capsule, tablets, pill, powder, or granule. In certain embodiments, the pharmaceutical composition is in the form of a sterilized pH buffered aqueous salt solution. In certain embodiments, the pharmaceutical composition is in the form of a container configured to spray a liquid or sealed container with a propellant.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands.

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

In certain embodiment, this disclosure contemplates pharmaceutical compositions comprising peptides disclosed herein, and agents disclosed herein and pharmaceutically acceptable excipient. In certain embodiments, this disclosure contemplates the production of a medicament comprising peptides disclosed herein, or agents disclosed herein and uses for methods disclosed herein.

### Methods of Treatment

In some aspects, disclosed herein is a method of treating an inflammatory disease (e.g., an autoimmune disease) in a subject in need thereof, comprising administering to the subject a therapeutically effective amount of the recombinant polypeptide, the recombinant polynucleotide, or the pharmaceutical composition of any preceding aspect. In some embodiments, the inflammatory disease comprises multiple sclerosis, graft-versus-host disease, transplant rejection reactions, asthma, type 1 diabetes (TID), alopecia areata, rheumatoid arthritis, ankylosing spondylitis, systemic lupus erythematosus (SLE), psoriasis, Behcet's disease, granulomatosis with polyangiitis, Takayasu's disease, Crohn's disease, ulcerative colitis, autoimmune hepatitis, sclerosing cholangitis, or sepsis.

In some embodiments, a desired therapeutic result is the control of the inflammatory disease. In some embodiments, a desired therapeutic result is reduction of levels of one or more proinflammatory cytokines in the treated subjects (e.g., reduced levels of one or more of IL-1, IL-6, and/or TNFα). In some embodiments, a desired therapeutic result is increased levels of Tregs. In some embodiments, a desired therapeutic result is decreased levels and/or proliferation of CD4_T cells producing IFNγ. In some aspects, disclosed herein is a method of enhancing immune responses to a cancer or an infectious disease in a subject in need thereof, comprising administering to the subject a therapeutically effective amount of the recombinant polypeptide, the recombinant polynucleotide, or the pharmaceutical composition of any preceding aspect.

In some aspects, disclosed herein is a method of treating a cancer or an infectious disease in a subject in need thereof, comprising administering to the subject a therapeutically effective amount of the recombinant polypeptide, the recombinant polynucleotide, or the pharmaceutical composition of any preceding aspect.

Ins some embodiments, the recombinant polypeptide comprises:
an IL-2 polypeptide;
a CD25 polypeptide; and
an IL-10 polypeptide.

In some embodiments, the CD25 polypeptide comprises an extracellular domain of a CD25 protein. In some embodiments, the IL-10 polypeptide is linked to the C-terminus of the CD25 polypeptide.

In some embodiments, the IL-2 polypeptide comprises a sequence at least 80% (for example, at least about 80%, about 85%, about 90%, about 95%, or about 98%) identical to SEQ ID NO: 1 or 7 or a fragment thereof. In some embodiments, the IL-2 polypeptide comprises a C145S mutation relative to SEQ ID NO: 7. In some embodiments, the IL-2 polypeptide comprises the sequence of SEQ ID NO: 4 or a fragment thereof.

In some embodiments, the CD25 polypeptide comprises a truncated C-terminus. In some embodiments, the CD25 polypeptide comprises a truncated C-terminus from residues 213 to 240 of the extracytoplasmic domain residues of CD25. In some embodiments, the C-terminus truncation refers to amino acid residues 213 to 240 relative to a wild type CD25. In some embodiments, the wild type CD25 polypeptide comprises the sequence set forth in SEQ ID NO: 22.

In some embodiments, the CD25 polypeptide comprises a sequence at least 80% (for example, at least about 80%, about 85%, about 90%, about 95%, or about 98%) identical to SEQ ID NO: 2 or 5 or a fragment thereof.

In some embodiments, the IL-10 polypeptide comprises a sequence at least 80% (for example, at least about 80%, about 85%, about 90%, about 95%, or about 98%) identical to SEQ ID NO: 3 or 6 or a fragment thereof.

In some embodiments, the recombinant polypeptide of any preceding aspect comprises a sequence at least 80% (for example, at least about 80%, about 85%, about 90%, about 95%, or about 98%) identical to SEQ ID NO: 8 or a fragment thereof.

In some embodiments, the recombinant polypeptide of any preceding aspect comprises a sequence at least 80% (for example, at least about 80%, about 85%, about 90%, about 95%, or about 98%) identical to SEQ ID NO: 9 or a fragment thereof.

In some aspects, the methods disclosed comprises administering to a subject in need a therapeutically effective amount of a recombinant polynucleotide encoding a recombinant polypeptide comprising:
an IL-2 polypeptide;
a CD25 polypeptide; and
an IL-10 polypeptide.

In some embodiments, the recombinant polynucleotide is at least 80% (for example, at least about 80%, about 85%, about 90%, about 95%, or about 98%) identical to SEQ ID NO: 10 or 17 or a fragment thereof. In some embodiments, the vector comprises an IL-12 polynucleotide at least 80% (for example, at least about 80%, about 85%, about 90%, about 95%, or about 98%) identical to SEQ ID NO: 12 or 19 or a fragment thereof. In some embodiments, the vector comprises a CD25 polynucleotide at least 80% (for example, at least about 80%, about 85%, about 90%, about 95%, or about 98%) identical to SEQ ID NO: 15 or 20 or a fragment thereof. In some embodiments, the vector comprises an IL-10 polynucleotide at least 80% (for example, at least about 80%, about 85%, about 90%, about 95%, or about 98%) identical to SEQ ID NO: 16 or 21 or a fragment thereof. In some embodiments, the recombinant polypeptide further comprises one or more linker sequences, wherein the linker sequence comprises a sequence at least 80% (for example, at least about 80%, about 85%, about 90%, about 95%, or about 98%) identical to SEQ ID NO: 13 or 14 or a fragment thereof. In some embodiments, the recombinant polypeptide further comprises a signal sequence, wherein the signal sequence comprises a sequence at least 80% (for example, at least about 80%, about 85%, about 90%, about 95%, or about 98%) identical to SEQ ID NO: 11 or 18 or a fragment thereof.

In some embodiments, the recombinant polynucleotide comprises a nucleic acid sequence at least 80% (for example, at least about 80%, about 85%, about 90%, about 95%, or about 98%) identical to SEQ ID NO: 10 or a fragment thereof.

In some embodiments, the recombinant polynucleotide comprises a nucleic acid sequence at least 80% (for example, at least about 80%, about 85%, about 90%, about 95%, or about 98%) identical to SEQ ID NO: 17 or a fragment thereof.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In some embodiments, a desired therapeutic result is reduction of tumor size or cancer cells. In some embodiments, a desired therapeutic result is prevention of recurrence. In some embodiments, a desired therapeutic result is prolonged survival. In some embodiments, a desired therapeutic result is increased levels of anti-tumor immune cells (e.g., cytotoxic CD8 T cells).

In some embodiments, the infectious disease is caused by a viral infection, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

In some embodiments, the infectious disease is caused by a bacterial infection, wherein the bacterial infection comprises an infection of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

In some embodiments, the infectious disease is caused by a fungal infection, wherein the fungal infection comprises an infection of Candida albicans, Cryptococcus neoformans, Histoplama capsulatum, Aspergillus fumigatus, Coccidiodes immitis, Paracoccidioides brasiliensis, Blastomyces dermitidis, Pneumocystis carinii, Penicillium marneffi, or Alternaria alternate.

In some embodiments, the infectious disease is caused by a parasitic infection, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

Dosing frequency for the composition disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

This section discusses strategies used to improve on IL-10 and to highlight the advantages of IL-2-10/CD25 bifunctional fusion proteins (FP). This FP expands Tregs via IL-2 activity while simultaneously limiting inflammation via IL-10 activity. Although IL-2 and IL-10 can promote immune responses, the dominant role of both cytokines is to regulate autoimmunity as demonstrated by IL-2 or IL-10 knockout mice. The design shown herein can avoid IL-2- or IL-10-dependent stimulatory effects on autoreactive T cells because low IL-2R signaling targets Tregs, not autoreactive T effector cells, and immune activation by IL-10 is related to the tumor, not autoimmune, microenvironments. Bifunctional IL2-10/CD25 can also favor targeting this FP to the Treg microenvironment due to high levels of the high affinity IL-2R on Tregs.

Low amounts of rIL-2 or agonist IL-2/anti-IL-2 complexes that target the high affinity IL-2R expand Tregs and limit autoimmunity in mouse models of diabetes, EAE, and islet allograft rejection and is now being tested in clinical trials (see Klatzmann and Abbas (2015) Nat. Rev. Immunol. 15:283-294 and Sharabi et al (2018) Nat. Rev. Drug Discov. 17:823-844). Low-dose IL-2 was first used in clinical trials for chronic GVHD and HCV-induced vasculitis. Such trials have also been completed for patients with type 1 diabetes (TID), alopecia areata, rheumatoid arthritis, ankylosing spondylitis, SLE, psoriasis, Behcet's disease, granulomatosis with polyangiitis, Takayasu's disease, Crohn's disease, ulcerative colitis, autoimmune hepatitis, and sclerosing cholangitis. From these trials, low-dose rIL-2 is ˜1 million IU of rIL-2 administered at frequencies ranging between daily to every other week. These trials also indicate that low-dose IL-2 therapy is safe, Tregs increase in all patients, but at variable levels, and many patients show clinical improvement, but not complete resolution of disease. Importantly, self-reactive T cells are not reactivated. This latter result reflects that human Tregs are at least 100-fold more responsive than CD4+CD45RO+ Teff cells to low amounts of rIL-2. Nevertheless, low-dose rIL-2 monotherapy is unlikely to restore immune tolerance as beneficial effects require continued administration of IL-2. An alternative idea, which has not previously been addressed, is to use IL-2 fusions in combination strategies to limit autoimmunity and perhaps restore immune tolerance.

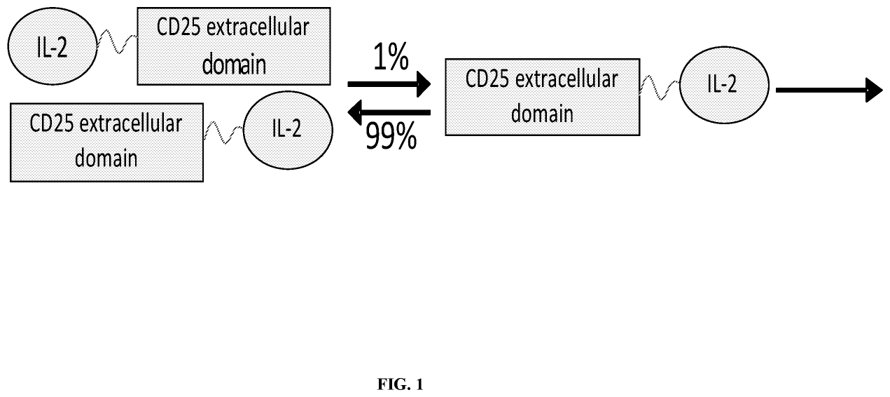

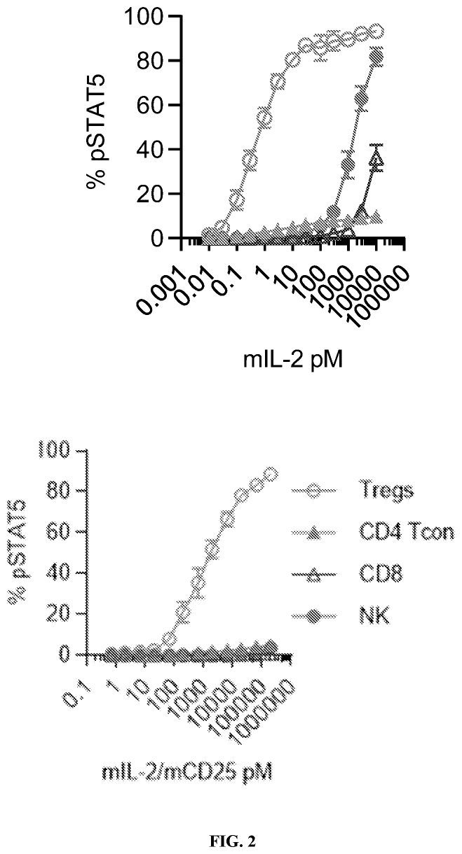

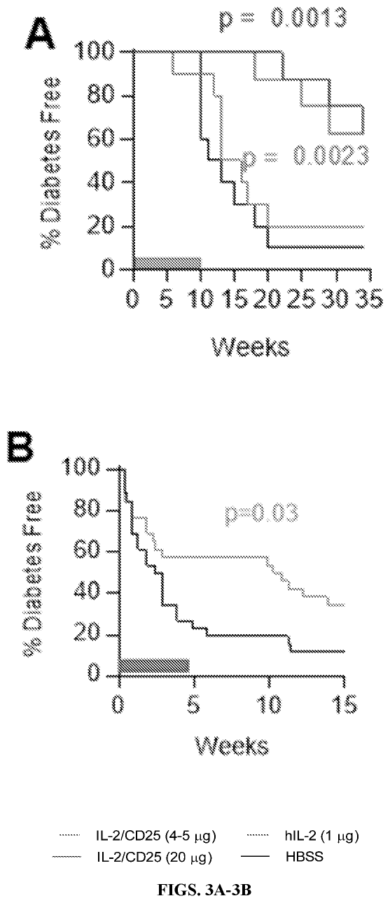

A FP that links IL-2 to CD25 (IL-2/CD25) has been developed (see Ward et al. (2018) J. Immunol. 201:2579-2592). This FP has a longer half-life (16-18 hr) than rIL-2 (˜10 min). Structurally, IL-2/CD25 forms inactivate non-covalent transdimers (˜111 kDa) that slowly dissociate into biologically active monomers (˜56 kDa) (FIG. 1) that at low dose stimulates cells such as Tregs and at high dose CD4+ and CD8+T effector cells that express the high affinity IL-2R. This selectivity of IL-2/CD25 is shown by its inability to activate STAT5 in memory-phenotypic CD8+T and NK cells that express the intermediate affinity IL-2R (FIG. 2) over a large range of concentrations. At a low dose (˜5 μg) of IL-2/CD25 in vivo, this mechanism leads to a persistent amount of IL-2 activity to support Tregs, but not Teff cells, the latter of which also express the high affinity IL-2R, but at lower levels than Tregs. Due to these properties of mIL-2/CD25, a similar amount of moles of IL-2/CD25, but not rIL-2, expanded Tregs in vivo and limited diabetes in pre-diabetic NOD mice (FIG. 3A) and delayed diabetes when administered to hyperglycemic mice (FIG. 3B). However, IL-2/CD25 did not fully protect all NOD mice from diabetes. Thus, these findings highlight a need for improving the efficacy of Treg-targeted mIL-2/CD25-dependent immunotherapy of autoimmunity. At a high dose IL-2/CD25 promotes anti-tumor immunity.

### Example 2. Bifunctional IL-2 and IL-10 Fusion Protein

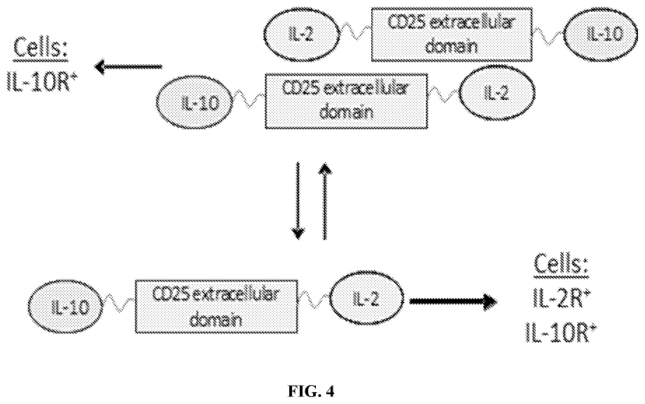

Bifunctional IL-2 and IL-10 FP: IL-2-10/CD25 FP combines IL-2-dependent Treg expansion with IL-10-dependent anti-inflammatory activity (FIG. 4). Of the many immunosuppressive cytokines, IL-10 was chosen because its non-redundant function is to limit inflammatory responses. Inflammation not only destabilizes Treg function but also lowers Treg responsiveness to IL-2. IL-10 directly acts on APCs to inhibit their production of inflammatory cytokines such as IL-1, IL-6, and TNF. IL-10 also lowers T cell activation directly through inhibition of IFNγ and anti-proliferative effects, especially on CD4+ T cells, and indirectly by inhibition of co-stimulatory and MHC molecules on APCs. Autoimmunity, particularly colitis, is associated with IL-10 deficiency in mouse and man. Correspondingly, in pre-clinical models, IL-10 limits colitis and diabetes. Treg production of IL-10 is essential to suppress T-cell-mediated colitis in mice. In addition, the selective deletion of the IL-10R in Tregs leads to colitis due to impaired Treg secretion of IL-10 and suppression of Th17 cells. IL-10R signaling in Tregs directly supports their survival and function. Thus, enhancing IL-10 activity in conjunction with increasing Tregs can lead to more effective control of autoimmunity directly by its anti-inflammatory activity and indirectly by enhancing Tregs. The clinical use of IL-10 to combat colitis and other inflammatory disorders has been disappointing. These failures can be due to its short half-life and/or inability to achieve therapeutic concentrations within the gut mucosa. Biologically active IL-10 is a non-covalent dimer but is unstable in vivo. IL-10 has been engineered to improve its pharmacokinetics and pharmacodynamics. Such IL-10s include IL-10-Ig fusion proteins, PEGylated IL-10 and covalently-dimerized IL-10 to increase its half-life, IL-10 muteins with enhanced affinity for the IL-10R in vitro, and bispecific fusions proteins linking IL-10 to IL-4 or IL-10 to anti-CD86 single chain Fv-IgG1. The IL-4/IL-10 FP broadens the anti-inflammatory effect whereas the IL-10/anti-CD86 FP selectively directs IL-10R signaling to APCs.

As described herein, a bi-specific fusion protein linking IL-2 and IL-10 to CD25 (IL-2-10/CD25) can be used to enhance Tregs and IL-10 in the context of autoimmunity. IL-2-10/CD25 can provide improved immunosuppression of autoreactive T cells by: 1) extending the half-life of both cytokines, 2) more effective enhancement of Treg suppressive mechanisms, and 3) optimizing IL-10-mediated inhibition of inflammatory responses. As IL-2/CD25 readily increases Tregs in the inflamed pancreas and the gut mucosa, IL-2-10/CD25 can localize the IL-10 component of this FP in the inflamed environment with Tregs and other closely associated cells, such as APCs and autoreactive T cells. In addition, the associated IL-10 moiety can exert direct inhibitory effects on cells that express high levels of the IL-10R, such as APCs, to favor tolerogenic DCs. Moreover, IL-2-10/CD25 may under some circumstances promote immunity to cancer and infectious disease as either cytokine alone also has activity in these settings.

This disclosure relates to targeting two complementary pathways simultaneously at one time with a single biologic. Low-dose mIL-2/CD25 is specific for Tregs and boosts their numbers and function. Coupling IL-10 activity to mIL-2/CD25 can further enhance Treg survival through direct action on Tregs and the associated IL-10 activity may promote immune tolerance by its inhibitory action on APCs and Teff cells. This mode of action has not been targeted with existing IL-10 biologics. It has been shown that mIL-2-10/CD25 has bifunctional activity.

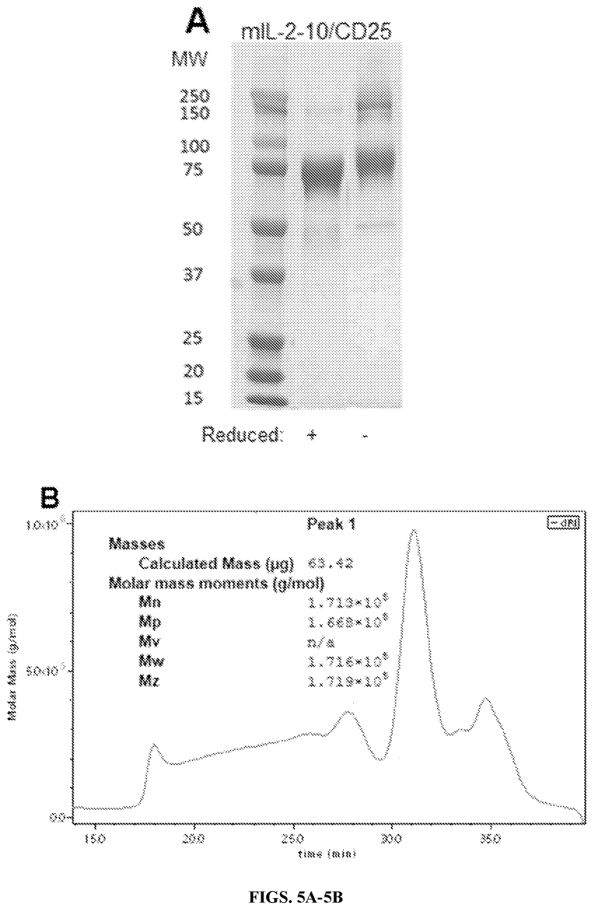

Development of mouse IL-2-10/CD25 (mIL-2-10/CD25): IL-10 is normally found as a dimer, but IL-10 monomers are biologically active, but with ˜10-fold lower activity (see Josephson (2000) J. Biol. Chem. 275:13352-133257). The attenuated activity of monomer IL-10 may promote selectivity toward cells with higher amounts of IL-10R. mIL-2/CD25 was engineered to contain IL-10. (FIG. 4). The C-terminus of mouse IL-2 was linked to the N-terminus of mCD25 through a glycine serine linker (G3S)₃ and the C-terminus of mouse CD25 was linked to the N-terminus of mouse IL-10 through a glycine serine linker (G3S) 3. To facilitate purification, a Gly₂His₆-tag was linked to the C-terminus of mouse IL-10. This design supports non-covalent IL-2-10/CD25 transdimers through IL-2 interactions in trans with CD25. These dimers can dissociate into monomers, where the monomer and dimer forms of mIL-2-10/CD25 can retain IL-10 activity but only the monomer form has IL-2 activity. In this latter configuration, this bifunctional molecule may also promote selectivity toward cells that co-express the IL-2R and IL-10R. Biochemical analyses showed that under denaturing and reducing conditions t mIL-2-10/CD25 exhibited a single major band of approximately 75 kDa, expected for a monomer of mIL-2-10/CD25 (FIG. 5A). However, under native conditions, mIL-2-10/CD25 was predominately a dimer, but also contained other higher molecular weight species and a significant amount of monomers (FIG. 5B).

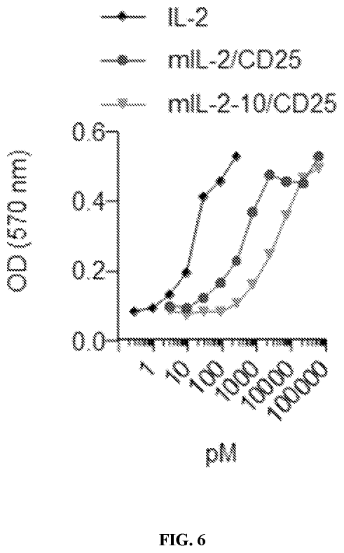

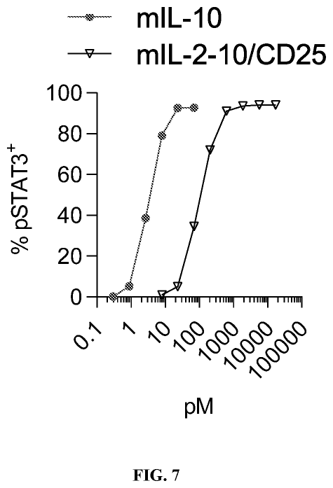

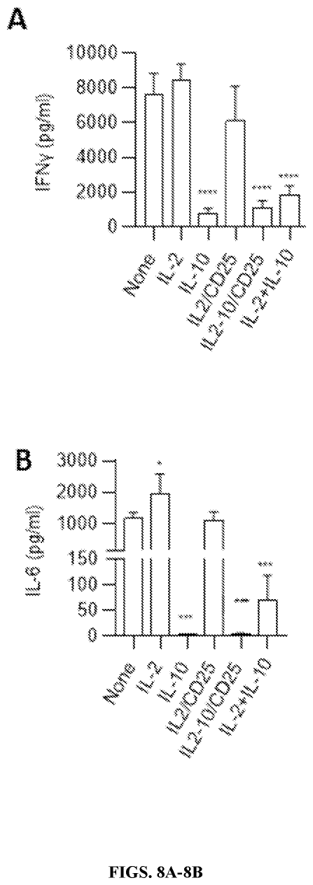

mIL-2/CD25 has low activity in vitro (˜100-fold lower than rIL-2), due to the predominant inactive transdimer form of the FP (FIG. 6), but has high activity in vivo, due to the steady dissociation of biologically active mIL-2/CD25 monomers (FIG. 1) Purified dimer mIL-2-10/CD25 has ˜10-fold less IL-2 activity than mIL-2/CD25, as assessed on the IL-2-dependent CTLL cell line (FIG. 6). This lower activity likely reflects an influence of IL-10 on the conformation of mIL-2/CD25 and/or slower dimer dissociation into active monomer. Purified dimer mIL-2-10/CD25 is ˜20-30-fold less active than rIL-10, as assessed by induction of pSTAT3 in the RAW 264.7 macrophage cell line (FIG. 7). This level of IL-10 activity is slightly lower than expected for IL-10 monomers. The IL-10 moiety of mIL-2-10/CD25 also led to the inhibition of anti-CD3-induced-IFNγ secretion by CD8+ T cells (FIG. 8A) and LPS-induced IL-6 production (FIG. 8B) with activity expected for an IL-10 monomer. These data indicate that this bifunctional FP has relatively high IL-10 activity where some of the IL-10 activity appears not to depend on dissociation of the transdimers.

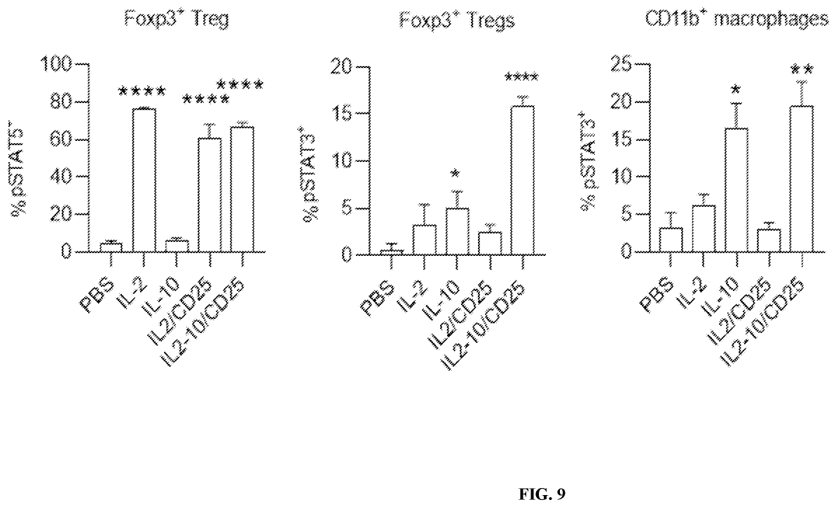

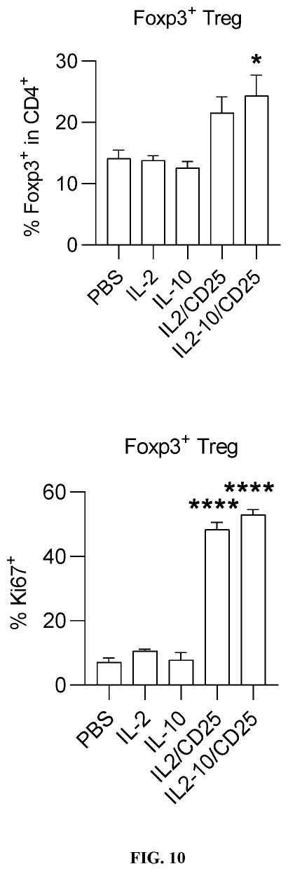

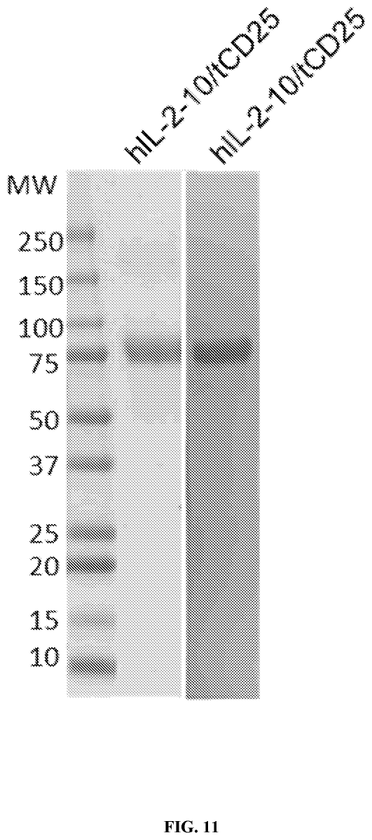

mIL-2-10/CD25 also exhibits bifunctional activity in vivo. At 90 minutes post-injection into C57BL/6 mice, substantial and similar amount of pSTAT5 was induced in Tregs by mIL-2/CD25 and mIL-2-10/CD25. mIL-2-10/CD25, but not mIL-2/CD25, activated pSTAT3 in Tregs and CD11b+ macrophages (FIG. 9). Thus, the IL-10 moiety of mIL-2-10/CD25 not only activates Tregs and APC but it was more effective than IL-10 in targeting Tregs, consistent with the ability to localize IL-10 activity of the bifunctional FP in the Treg microenvironment. mIL-2-10/CD25 (50 μg) retained IL-2-dependent selectivity toward Tregs as pSTAT5 was not detected in CD8+ T cells that express the intermediate affinity IL-2R. At 72 hr post-injection, mIL-2/CD25 and mIL-2-10/CD25 led to similar Treg expansion, but this occurred using 10-fold greater amounts of mIL-2-10/CD25 (FIG. 10). mIL-2-10/CD25 has approximately 10-fold lower IL-2 activity than mIL-2/CD25 when assessed in vitro (FIG. 5A). Overall, 50 μg of mIL-2-10/CD25 represents a Treg selective amount of this FP while retaining high IL-10 activity to limit inflammatory responses.

### Example 3. Development of Less Aggregated Human IL-2-10/CD25 (hIL-2-10/tCD25)

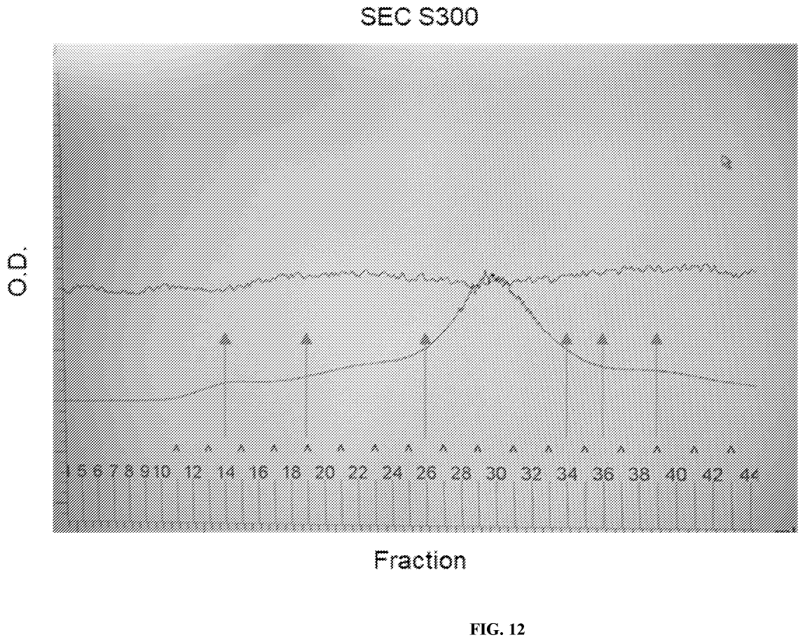

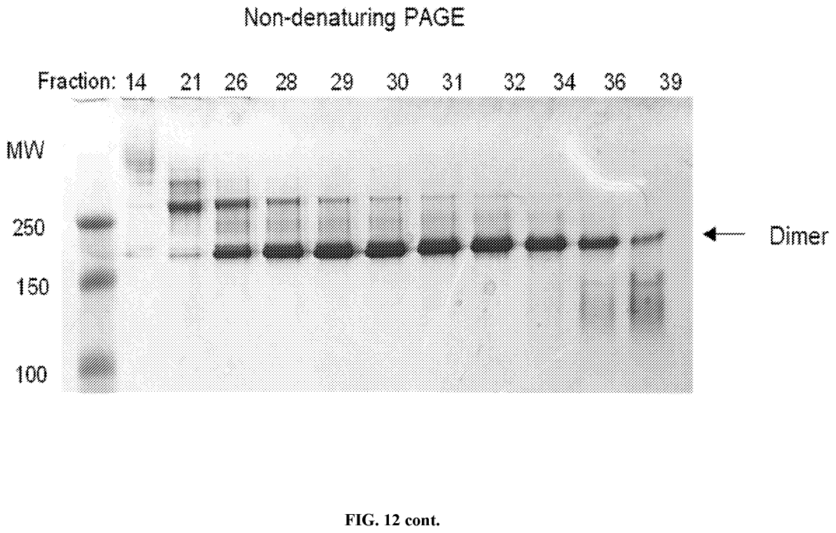

hIL-2-10/CD25 was engineered to limit aggregation. The free cysteine in human IL-2 at residue 143 was mutated to serine and the free cysteine in the ectodomain of human CD25 was eliminated by truncation of residues 213-240. The C-terminus of human IL-2 was linked to the N-terminus of truncated human CD25 (tCD25) through a glycine serine linker (G3S) 3 and the C-terminus of tCD25 was linked to the N-terminus of human IL-10 through a glycine serine linker (G3S)₃. To facilitate purification, a Gly₂His₆-tag was linked to the C-terminus of human IL-10. Purified hIL-2-10t/CD25 by SDS-PAGE revealed a single expected band of approximately 75 kDa (FIG. 11). Analysis under native conditions showed a single major band consistent with dimers, with minimal detection of monomers or higher order multimers (FIG. 12). Thus, engineered hIL-2-10/tCD25 exhibited reduced the heterogeneity when compared to mIL-2-10/CD25 (FIG. 5B).

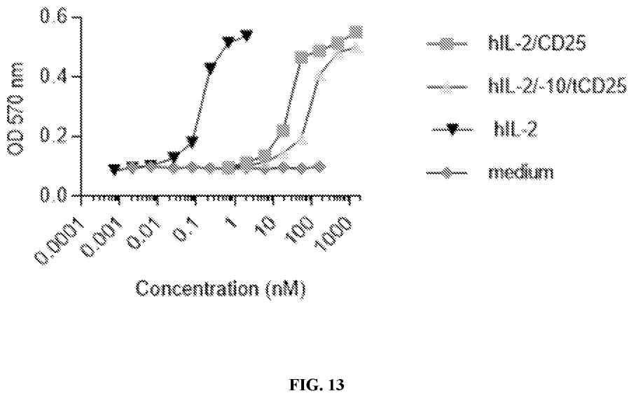

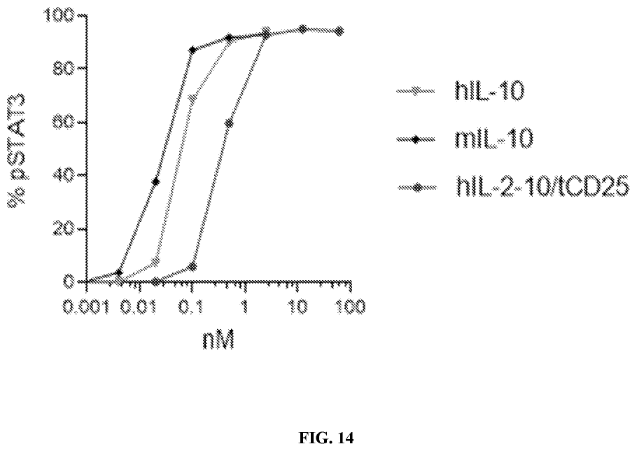

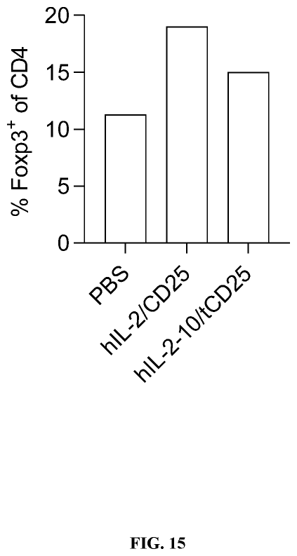

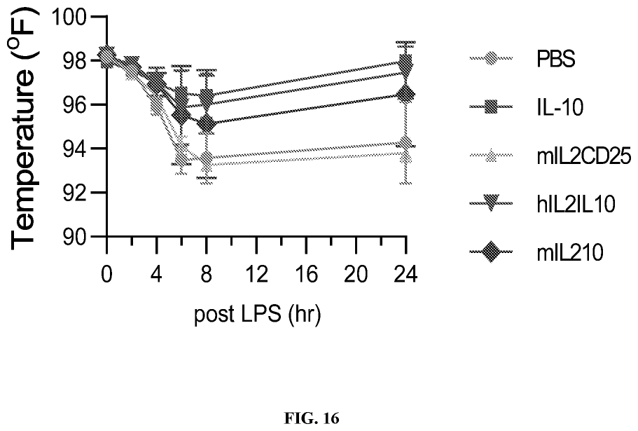

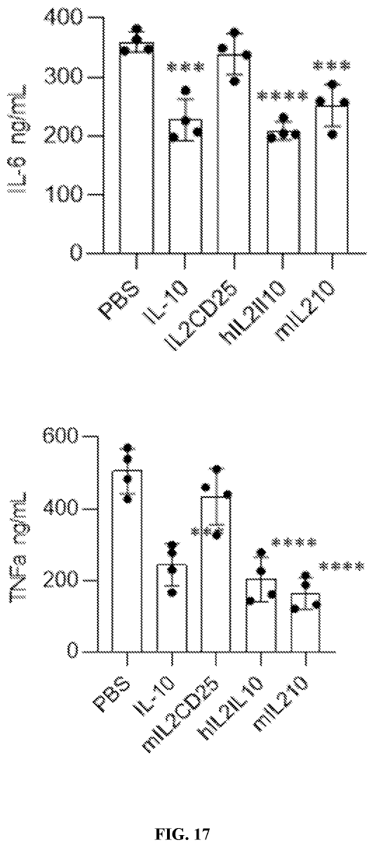

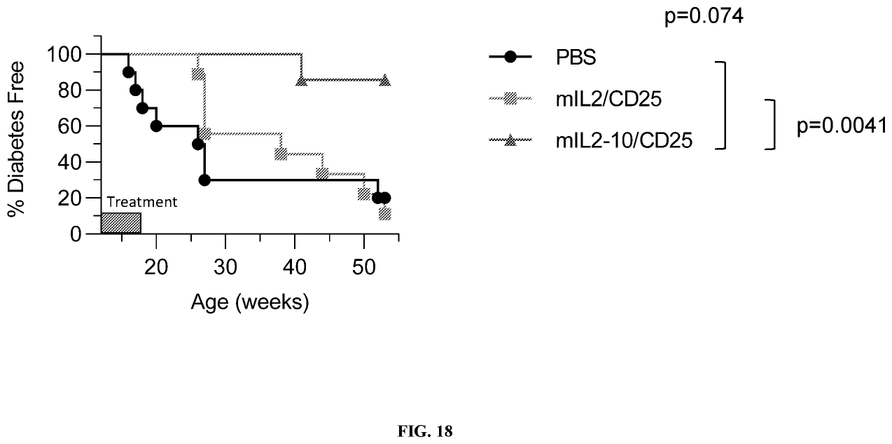

In vitro functional studies indicated that purified dimer hIL-2-10/tCD25 exhibited IL-2 (FIG. 13) and IL-10 activity (FIG. 14) in a manner analogous to mIL-2-10/CD25. Treatment of mice with hIL-2-10/tCD25 increased the proportions of Tregs in the spleen (FIG. 15). Moreover, when mice received LPS, hIL-2-10/tCD25 limited the associated reduction in body temperature in a manner similar to recombinant IL-10 (FIG. 16). Under these conditions, hIL-2-10/tCD25 and IL-10 reduced the serum concentrations of IL-6 and TNFα (FIG. 17). Thus, these data indicate that hIL-2-10/tCD25 exhibits bifunctional activity in vivo.

### Example 4: MIL-2-10/CD25 Shows Superior Ability to Limit Autoimmunity

NOD mice with rapid acute diabetes are often refractory to therapeutic interventions (see Mathews et al (2015) Diabetes 64:3885-3890), including mIL-2/CD25 (FIG. 3b). Therefore, mIL-2/CD25 was compared to mIL-2-10/CD25 to limit diabetes in NOD mice with more slowly developing progressive diabetes. 12-week-old NOD mice with inflamed islets were treated with PBS, (control), mIL-2/CD25 (5 μg), and mIL-2-10/CD25 (50 μg) for 5 weeks. Since mIL-2-10/CD25 has 10-fold lower IL-2 activity when compared to mIL-2/CD25 (FIG. 6), 50 μg of mIL-2-10/CD25 was used as this dose achieves an expansion of Tregs similar to that induced by mIL-2/CD25 (FIG. 10). Progressive diabetes was determined by excluding mice that became diabetes between 12-15 weeks of age. This study revealed that mIL-2-10/CD25 was much more effective than mIL-2/CD25 in limiting diabetes as 6 of 7 mice remain diabetes free 36 weeks post-therapy. Thus, these data indicate that mIL-2-10/CD25 more effectively controls autoimmunity than mIL-2/CD25, here using a pre-clinical model of type 1 diabetes.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES (m = mouse; h = human)
mIL-2, unprocessed, SEQ ID NO: 1, Protein
MYSMQLASCVTLTLVLLVNSAPTSSPTSSPTSSSTAEAQQQQQQQQHLEQLLMDL
QELLSRMENYRNLKLPRMLTFKFYLPKQATELKDLQCLEDELGPLRHVLDLTQSK
SFQLEDAENFISNIRVTVVKLKGSDNTFECQFDDESATVVDFLRRWIAFCQSIIS
TSPQ
mCD25, extracytoplasmic domain, SEQ ID NO: 2, Protein
ELCLYDPPEVPNATFKALSYKNGTILNCECKRGFRRLKELVYMRCLGNSWSSNCQ
CTSNSHDKSRKQVTAQLEHQKEQQTTTDMQKPTQSMHQENLIGHCREPPPWKHED
SKRIYHFVEGQSVHYECIPGYKALQRGPAISICKMKCGKTGWTQPQLTCVDEREH
HRFLASEESQGSRNSSPESETSCPITTTDFPQPTETTAMTETFVLTMEYK
mIL-10, mature form, SEQ ID NO: 3, Protein
SRGQYSREDNNCTHFPVGQSHMLLELRTAFSQVKTFFQTKDQLDNILLTDSLMQD
FKGYLGCQALSEMIQFYLVEVMPQAEKHGPEIKEHLNSLGEKLKTLRMRLRRCHR
FLPCENKSKAVEQVKSDFNKLQDQGVYKAMNEFDIFINCIEAYMMIKMKS
hIL-2, unprocessed with C to S mutation, SEQ ID NO: 4, Protein
MYRMQLLSCIALSLALVTNSAPTSSSTKKTQLQLEHLLLDLQMILNGINNYKNPK
LTRMLTFKFYMPKKATELKHLQCLEEELKPLEEVLNLAQSKNFHLRPRDLISNIN
VIVLELKGSETTFMCEYADETATIVEFLNRWITFSQSIISTLT
hCD25, truncated extracytoplasmic domain, SEQ ID NO: 5, Protein
ELCDDDPPEIPHATFKAMAYKEGTMLNCECKRGFRRIKSGSLYMLCTGNSSHSSW
DNQCQCTSSATRNTTKQVTPQPEEQKERKTTEMQSPMQPVDQASLPGHCREPPPW
ENEATERIYHFVVGQMVYYQCVQGYRALHRGPAESVCKMTHGKTRWTQPQLICTG
EMETSQFPGEEKPQASPEGRPESETS
hIL-10, mature form, SEQ ID NO: 6, Protein
SPGQGTQSENSCTHFPGNLPNMLRDLRDAFSRVKTFFQMKDQLDNLLLKESLLED
FKGYLGCQALSEMIQFYLEEVMPQAENQDPDIKAHVNSLGENLKTLRLRLRRCHR
FLPCENKSKAVEQVKNAFNKLQEKGIYKAMSEFDIFINYIEAYMTMKIRN
hIL-2, unprocessed, SEQ ID NO: 7, Protein
MYRMQLLSCIALSLALVTNSAPTSSSTKKTQLQLEHLLLDLQMILNGINNYKNPK
LTRMLTFKFYMPKKATELKHLQCLEEELKPLEEVLNLAQSKNFHLRPRDLISNIN
VIVLELKGSETTFMCEYADETATIVEFLNRWITFCQSIISTLT
mIL2-linker-CD25-linker-IL10 (SEQ ID NO: 8), Protein
MYSMQLASCVTLTLVLLVNSAPTSSPTSSPTSSSTAEAQQQQQQQQHLEQLLMDL
QELLSRMENYRNLKLPRMLTFKFYLPKQATELKDLQCLEDELGPLRHVLDLTQSK
SFQLEDAENFISNIRVTVVKLKGSDNTFECQFDDESATVVDFLRRWIAFCQSIIS
TSPQGGGSGGGSGGGSELCLYDPPEVPNATFKALSYKNGTILNCECKRGFRRLKE
LVYMRCLGNSWSSNCQCTSNSHDKSRKQVTAQLEHQKEQQTTTDMQKPTQSMHQE
NLTGHCREPPPWKHEDSKRIYHFVEGQSVHYECIPGYKALQRGPAISICKMKCGK
TGWTQPQLTCVDEREHHRFLASEESQGSRNSSPESETSCPITTTDFPQPTETTAM
TETFVLTMEYKGGGSGGGSGGGSSRGQYSREDNNCTHFPVGQSHMLLELRTAFSQ
VKTFFQTKDQLDNILLTDSLMQDFKGYLGCQALSEMIQFYLVEVMPQAEKHGPEI
KEHLNSLGEKLKTLRMRLRRCHRFLPCENKSKAVEQVKSDFNKLQDQGVYKAMNE
FDIFINCIEAYMMIKMKSGGHHHHHH
Underlined: signal peptide
In bold: linkers and His 6x
hIL2-linker-CD25truncated-linker-IL10 (SEQ ID NO: 9), Protein
MYRMQLLSCIALSLALVTNSAPTSSSTKKTQLQLEHLLLDLQMILNGINNYKNPK
LTRMLTFKFYMPKKATELKHLQCLEEELKPLEEVLNLAQSKNFHLRPRDLISNIN
VIVLELKGSETTFMCEYADETATIVEFLNRWITFSQSIISTLTGGGSGGGSGGGS
ELCDDDPPEIPHATFKAMAYKEGTMLNCECKRGFRRIKSGSLYMLCTGNSSHSSW
DNQCQCTSSATRNTTKQVTPQPEEQKERKTTEMQSPMQPVDQASLPGHCREPPPW
ENEATERIYHFVVGQMVYYQCVQGYRALHRGPAESVCKMTHGKTRWTQPQLICTG
EMETSQFPGEEKPQASPEGRPESETSGGGSGGGSGGGSSPGQGTQSENSCTHFPG
NLPNMLRDLRDAFSRVKTFFQMKDQLDNLLLKESLLEDFKGYLGCQALSEMIQFY
LEEVMPQAENQDPDIKAHVNSLGENLKTLRLRLRRCHRFLPCENKSKAVEQVKNA
FNKLQEKGIYKAMSEFDIFINYIEAYMTMKIRNGGHHHHHH
Underlined: signal peptide
In bold: mutation C-S, linkers and His 6x
mIL-2-Linker-CD25-Linker-mIL-10 (SEQ ID NO: 10), DNA
ATGTACAGCATGCAGCTCGCATCCTGTGTCACACTGACACTTGTGCTCCTTGTCA
ACAGCGCACCCACTTCAAGCCCCACTTCAAGCCCCACTTCAAGCTCTACAGCGGA
AGCACAGCAGCAGCAGCAGCAGCAGCAGCACCTGGAGCAGCTGTTGATGGACCTA
CAGGAGCTCCTGAGCAGGATGGAGAATTACAGGAACCTGAAACTCCCCAGGATGC
TCACCTTCAAATTTTACTTGCCCAAGCAGGCCACAGAATTGAAAGATCTTCAGTG
CCTAGAAGATGAACTTGGACCTCTGCGGCATGTTCTGGATTTGACTCAAAGCAAA
AGCTTTCAATTGGAAGATGCTGAGAATTTCATCAGCAATATCAGAGTAACTGTTG
TAAAACTAAAGGGCTCTGACAACACATTTGAGTGCCAATTCGATGATGAGTCAGC
AACTGTGGTGGACTTTCTGAGGAGATGGATAGCCTTCTGTCAAAGCATCATCTCA
ACAAGCCCTCAAGGTGGAGGTTCTGGTGGAGGTTCTGGTGGAGGTTCTGAACTGT
GTCTGTATGACCCACCCGAGGTCCCCAATGCCACATTCAAAGCCCTCTCCTACAA
GAACGGCACCATCCTAAACTGTGAATGCAAGAGAGGTTTCCGAAGACTAAAGGAA
TTGGTCTATATGCGTTGCTTAGGAAACTCCTGGAGCAGCAACTGCCAGTGCACCA
GCAACTCCCATGACAAATCGAGAAAGCAAGTTACAGCTCAACTTGAACACCAGAA
AGAGCAACAAACCACAACAGACATGCAGAAGCCAACACAGTCTATGCACCAAGAG
AACCTTACAGGTCACTGCAGGGAGCCACCTCCTTGGAAACATGAAGATTCCAAGA
GAATCTATCATTTCGTGGAAGGACAGAGTGTTCACTACGAGTGTATTCCGGGATA
CAAGGCTCTACAGAGAGGTCCTGCTATTAGCATCTGCAAGATGAAGTGTGGGAAA
ACGGGGTGGACTCAGCCCCAGCTCACATGTGTAGATGAAAGAGAACACCACCGAT
TTCTGGCTAGTGAGGAATCTCAAGGAAGCAGAAATTCTTCTCCCGAGAGTGAGAC
TTCCTGCCCCATAACCACCACAGACTTCCCACAACCCACAGAAACAACTGCAATG
ACGGAGACATTTGTGCTCACAATGGAGTATAAGGGTGGAGGTTCTGGTGGAGGTT
CTGGTGGAGGTTCTAGCAGGGGCCAGTACAGCCGGGAAGACAATAACTGCACCCA
CTTCCCAGTCGGCCAGAGCCACATGCTCCTAGAGCTGCGGACTGCCTTCAGCCAG
GTGAAGACTTTCTTTCAAACAAAGGACCAGCTGGACAACATACTGCTAACCGACT
CCTTAATGCAGGACTTTAAGGGTTACTTGGGTTGCCAAGCCTTATCGGAAATGAT
CCAGTTTTACCTGGTAGAAGTGATGCCCCAGGCAGAGAAGCATGGCCCAGAAATC
AAGGAGCATTTGAATTCCCTGGGTGAGAAGCTGAAGACCCTCAGGATGCGGCTGA
GGCGCTGTCATCGATTTCTCCCCTGTGAAAATAAGAGCAAGGCAGTGGAGCAGGT
GAAGAGTGATTTTAATAAGCTCCAAGACCAAGGTGTCTACAAGGCCATGAATGAA
TTTGACATCTTCATCAACTGCATAGAAGCATACATGATGATCAAAATGAAAAGCG
GTGGACATCACCATCACCATCACGGGCCCGGGTAA
Underlined: Signal peptide: SEQ ID NO: 11, DNA
ATGTACAGCATGCAGCTCGCATCCTGTGTCACACTGACACTTGTGCTCCTTGTCA
ACAGC
mIL-2: SEQ ID NO: 12, DNA
GCACCCACTTCAAGCCCCACTTCAAGCCCCACTTCAAGCTCTACAGCGGAAGCAC
AGCAGCAGCAGCAGCAGCAGCAGCACCTGGAGCAGCTGTTGATGGACCTACAGGA
GCTCCTGAGCAGGATGGAGAATTACAGGAACCTGAAACTCCCCAGGATGCTCACC
TTCAAATTTTACTTGCCCAAGCAGGCCACAGAATTGAAAGATCTTCAGTGCCTAG
AAGATGAACTTGGACCTCTGCGGCATGTTCTGGATTTGACTCAAAGCAAAAGCTT
TCAATTGGAAGATGCTGAGAATTTCATCAGCAATATCAGAGTAACTGTTGTAAAA
CTAAAGGGCTCTGACAACACATTTGAGTGCCAATTCGATGATGAGTCAGCAACTG
TGGTGGACTTTCTGAGGAGATGGATAGCCTTCTGTCAAAGCATCATCTCAACAAG
CCCTCAA
Linker 1: SEQ ID NO: 13, DNA
GGTGGAGGTTCTGGTGGAGGTTCTGGTGGAGGTTCT
Linker 2: SEQ ID NO: 14, DNA
CCCGGG
mCD25: SEQ ID NO: 15, DNA
GAACTGTGTCTGTATGACCCACCCGAGGTCCCCAATGCCACATTCAAAGCCCTCT
CCTACAAGAACGGCACCATCCTAAACTGTGAATGCAAGAGAGGTTTCCGAAGACT
AAAGGAATTGGTCTATATGCGTTGCTTAGGAAACTCCTGGAGCAGCAACTGCCAG
TGCACCAGCAACTCCCATGACAAATCGAGAAAGCAAGTTACAGCTCAACTTGAAC
ACCAGAAAGAGCAACAAACCACAACAGACATGCAGAAGCCAACACAGTCTATGCA
CCAAGAGAACCTTACAGGTCACTGCAGGGAGCCACCTCCTTGGAAACATGAAGAT
TCCAAGAGAATCTATCATTTCGTGGAAGGACAGAGTGTTCACTACGAGTGTATTC
CGGGATACAAGGCTCTACAGAGAGGTCCTGCTATTAGCATCTGCAAGATGAAGTG
TGGGAAAACGGGGTGGACTCAGCCCCAGCTCACATGTGTAGATGAAAGAGAACAC
CACCGATTTCTGGCTAGTGAGGAATCTCAAGGAAGCAGAAATTCTTCTCCCGAGA
GTGAGACTTCCTGCCCCATAACCACCACAGACTTCCCACAACCCACAGAAACAAC
TGCAATGACGGAGACATTTGTGCTCACAATGGAGTATAAG
mIL-10: SEQ ID NO: 16, DNA
AGCAGGGGCCAGTACAGCCGGGAAGACAATAACTGCACCCACTTCCCAGTCGGCC
AGAGCCACATGCTCCTAGAGCTGCGGACTGCCTTCAGCCAGGTGAAGACTTTCTT
TCAAACAAAGGACCAGCTGGACAACATACTGCTAACCGACTCCTTAATGCAGGAC
TTTAAGGGTTACTTGGGTTGCCAAGCCTTATCGGAAATGATCCAGTTTTACCTGG
TAGAAGTGATGCCCCAGGCAGAGAAGCATGGCCCAGAAATCAAGGAGCATTTGAA
TTCCCTGGGTGAGAAGCTGAAGACCCTCAGGATGCGGCTGAGGCGCTGTCATCGA
TTTCTCCCCTGTGAAAATAAGAGCAAGGCAGTGGAGCAGGTGAAGAGTGATTTTA
ATAAGCTCCAAGACCAAGGTGTCTACAAGGCCATGAATGAATTTGACATCTTCAT
CAACTGCATAGAAGCATACATGATGATCAAAATGAAAAGCGGTGGACATCACCAT
CACCATCACGGG
hIL2-linker-CD25truncated-linker-IL10 (SEQ ID NO: 17), DNA
ATGTACAGGATGCAACTCCTGTCTTGCATTGCACTAAGTCTTGCACTTGTCACAA
ACAGTGCACCTACTTCAAGTTCTACAAAGAAAACACAGCTACAACTGGAGCATTT
ACTGCTGGATTTACAGATGATTTTGAATGGAATTAATAATTACAAGAATCCCAAA
CTCACCAGGATGCTCACATTTAAGTTTTACATGCCCAAGAAGGCCACAGAACTGA
AACATCTTCAGTGTCTAGAAGAAGAACTCAAACCTCTGGAGGAAGTGCTAAATTT
AGCTCAAAGCAAAAACTTTCACTTAAGACCCAGGGACTTAATCAGCAATATCAAC
GTAATAGTTCTGGAACTAAAGGGATCTGAAACAACATTCATGTGTGAATATGCTG
ATGAGACAGCAACCATTGTAGAATTTCTGAACAGATGGATTACCTTTTGTCAAAG
CATCATCTCAACACTAACTGGTGGAGGTTCTGGTGGAGGTTCTGGTGGAGGTTCT
GAGCTCTGTGACGATGACCCGCCAGAGATCCCACACGCCACATTCAAAGCCATGG
CCTACAAGGAAGGAACCATGTTGAACTGTGAATGCAAGAGAGGTTTCCGCAGAAT
AAAAAGCGGGTCACTCTATATGCTCTGTACAGGAAACTCTAGCCACTCGTCCTGG
GACAACCAATGTCAATGCACAAGCTCTGCCACTCGGAACACAACGAAACAAGTGA
CACCTCAACCTGAAGAACAGAAAGAAAGGAAAACCACAGAAATGCAAAGTCCAAT
GCAGCCAGTGGACCAAGCGAGCCTTCCAGGTCACTGCAGGGAACCTCCACCATGG
GAAAATGAAGCCACAGAGAGAATTTATCATTTCGTGGTGGGGCAGATGGTTTATT
ATCAGTGCGTCCAGGGATACAGGGCTCTACACAGAGGTCCTGCTGAGAGCGTCTG
CAAAATGACCCACGGGAAGACAAGGTGGACCCAGCCCCAGCTCATATGCACAGGT
GAAATGGAGACCAGTCAGTTTCCAGGTGAAGAGAAGCCTCAGGCAAGCCCCGAAG
GCCGTCCTGAGAGTGAGACTTCCGGTGGAGGTTCTGGTGGAGGTTCTGGTGGAGG
TTCTAGCCCAGGCCAGGGCACCCAGTCTGAGAACAGCTGCACCCACTTCCCAGGC
AACCTGCCTAACATGCTTCGAGATCTCCGAGATGCCTTCAGCAGAGTGAAGACTT
TCTTTCAAATGAAGGATCAGCTGGACAACTTGTTGTTAAAGGAGTCCTTGCTGGA
GGACTTTAAGGGTTACCTGGGTTGCCAAGCCTTGTCTGAGATGATCCAGTTTTAC
CTGGAGGAGGTGATGCCCCAAGCTGAGAACCAAGACCCAGACATCAAGGCGCATG
TGAACTCCCTGGGGGAGAACCTGAAGACCCTCAGGCTGAGGCTACGGCGCTGTCA
TCGATTTCTTCCCTGTGAAAACAAGAGCAAGGCCGTGGAGCAGGTGAAGAATGCC
TTTAATAAGCTCCAAGAGAAAGGCATCTACAAAGCCATGAGTGAGTTTGACATCT
TCATCAACTACATAGAAGCCTACATGACAATGAAGATACGAAAC
Underlined: Signal peptide SEQ ID NO: 18, DNA
ATGTACAGGATGCAACTCCTGTCTTGCATTGCACTAAGTCTTGCACTTGTCAC
AAACAGT
hIL-2, SEQ ID NO: 19, DNA
GCACCTACTTCAAGTTCTACAAAGAAAACACAGCTACAACTGGAGCATTTACTGC
TGGATTTACAGATGATTTTGAATGGAATTAATAATTACAAGAATCCCAAACTCAC
CAGGATGCTCACATTTAAGTTTTACATGCCCAAGAAGGCCACAGAACTGAAACAT
CTTCAGTGTCTAGAAGAAGAACTCAAACCTCTGGAGGAAGTGCTAAATTTAGCTC
AAAGCAAAAACTTTCACTTAAGACCCAGGGACTTAATCAGCAATATCAACGTAAT
AGTTCTGGAACTAAAGGGATCTGAAACAACATTCATGTGTGAATATGCTGATGAG
ACAGCAACCATTGTAGAATTTCTGAACAGATGGATTACCTTTTGTCAAAGCATCA
TCTCAACACTAACT
hCD25 truncated, SEQ ID NO: 20, DNA
GAGCTCTGTGACGATGACCCGCCAGAGATCCCACACGCCACATTCAAAGCCATGG
CCTACAAGGAAGGAACCATGTTGAACTGTGAATGCAAGAGAGGTTTCCGCAGAAT
AAAAAGCGGGTCACTCTATATGCTCTGTACAGGAAACTCTAGCCACTCGTCCTGG
GACAACCAATGTCAATGCACAAGCTCTGCCACTCGGAACACAACGAAACAAGTGA
CACCTCAACCTGAAGAACAGAAAGAAAGGAAAACCACAGAAATGCAAAGTCCAAT
GCAGCCAGTGGACCAAGCGAGCCTTCCAGGTCACTGCAGGGAACCTCCACCATGG
GAAAATGAAGCCACAGAGAGAATTTATCATTTCGTGGTGGGGCAGATGGTTTATT
ATCAGTGCGTCCAGGGATACAGGGCTCTACACAGAGGTCCTGCTGAGAGCGTCTG
CAAAATGACCCACGGGAAGACAAGGTGGACCCAGCCCCAGCTCATATGCACAGGT
GAAATGGAGACCAGTCAGTTTCCAGGTGAAGAGAAGCCTCAGGCAAGCCCCGAAG
GCCGTCCTGAGAGTGAGACTTCC
hIL-10, SEQ ID NO: 21, DNA
AGCCCAGGCCAGGGCACCCAGTCTGAGAACAGCTGCACCCACTTCCCAGGCAACC
TGCCTAACATGCTTCGAGATCTCCGAGATGCCTTCAGCAGAGTGAAGACTTTCTT
TCAAATGAAGGATCAGCTGGACAACTTGTTGTTAAAGGAGTCCTTGCTGGAGGAC
TTTAAGGGTTACCTGGGTTGCCAAGCCTTGTCTGAGATGATCCAGTTTTACCTGG
AGGAGGTGATGCCCCAAGCTGAGAACCAAGACCCAGACATCAAGGCGCATGTGAA
CTCCCTGGGGGAGAACCTGAAGACCCTCAGGCTGAGGCTACGGCGCTGTCATCGA
TTTCTTCCCTGTGAAAACAAGAGCAAGGCCGTGGAGCAGGTGAAGAATGCCTTTA
ATAAGCTCCAAGAGAAAGGCATCTACAAAGCCATGAGTGAGTTTGACATCTTCAT
CAACTACATAGAAGCCTACATGACAATGAAGATACGAAAC
Full length hCD25 wild type, SEQ ID NO: 22, Protein
MDSYLLMWGLLTFIMVPGCQAELCDDDPPEIPHATFKAMAYKEGTMLNCECKRGF
RRIKSGSLYMLCTGNSSHSSWDNQCQCTSSATRNTTKQVTPQPEEQKERKTTEMQ
SPMQPVDQASLPGHCREPPPWENEATERIYHFVVGQMVYYQCVQGYRALHRGPAE
SVCKMTHGKTRWTQPQLICTGEMETSQFPGEEKPQASPEGRPESETSCLVTTTDF
QIQTEMAATMETSIFTTEYQVAVAGCVFLLISVLLLSGLTWQRRQRKSRRTI

1. A recombinant polypeptide comprising
an IL-2 polypeptide;
a CD25 polypeptide; and
an IL-10 polypeptide.

2. The recombinant polypeptide of claim 1, wherein the CD25 polypeptide comprises an extracellular domain of a CD25 protein.

3. The recombinant polypeptide of claim 1, wherein the IL-10 polypeptide is linked to the C-terminus of the CD25 polypeptide.

4. The recombinant polypeptide of claim 1, wherein the IL-2 polypeptide comprises a sequence at least 80% identical to SEQ ID NO: 1 or 7 or a fragment thereof.

5. The recombinant polypeptide of claim 1, wherein the IL-2 polypeptide comprises a C145S mutation relative to SEQ ID NO: 7.

6. The recombinant polypeptide of claim 5, wherein the IL-2 polypeptide comprises the sequence of SEQ ID NO: 4 or a fragment thereof.

7. The recombinant polypeptide of claim 1, wherein the CD25 polypeptide comprises a truncated C-terminus.

8. The recombinant polypeptide of claim 7, wherein CD25 polypeptide comprising the truncated C-terminus lacks amino acid residues 213 to 240 relative to a wild type CD25.

9. The recombinant polypeptide of claim 1, wherein the CD25 polypeptide comprises a sequence at least 80% identical to SEQ ID NO: 2 or 5 or a fragment thereof.

10. The recombinant polypeptide of claim 1, wherein the IL-10 polypeptide comprises a sequence at least 80% identical to SEQ ID NO: 3 or 6 or a fragment thereof.

11. The recombinant polypeptide of claim 1, comprising a sequence at least 80% identical to SEQ ID NO: 8 or 9 or a fragment thereof.

12. A recombinant polynucleotide comprising a nucleic acid sequence encoding the recombinant polypeptide of claim 1.

13. The recombinant polynucleotide of claim 12, wherein the nucleic acid sequence is at least 80% identical to SEQ ID NO: 10 or 17 or a fragment thereof.

14. A vector comprising the recombinant polynucleotide of claim 12.

15. A method of treating an inflammatory disease in a subject in need thereof, comprising administering to the subject a therapeutically effective amount of the recombinant polypeptide of claim 1.

16. The method of claim 15, wherein the inflammatory disease comprises systemic lupus erythematosus (SLE), multiple sclerosis, Addison disease, graft-versus-host disease, transplant rejection reactions, asthma, type 1 diabetes (TID), alopecia areata, rheumatoid arthritis, ankylosing spondylitis, psoriasis, Behcet's disease, granulomatosis with polyangiitis, Takayasu's disease, Crohn's disease, ulcerative colitis, Grave's disease, Hashimoto thyroiditis, myasthenia gravis, Sjogren syndrome, Celiac disease, pernicious anemia, psoriatic arthritis, autoimmune hepatitis, sclerosing cholangitis, Bullous pemphigoid, Juvenile idiopathic arthritis, scleroderma, hemolytic anemia, systemic sclerosis, Pemphigas, Gougerot-sjogrens, macrophage activating syndrome, Alzheimer's disease, myocarditis, or sepsis.

17. A method of enhancing immune responses to a cancer in a subject in need thereof, comprising administering to the subject a therapeutically effective amount of the recombinant polypeptide of claim 1.

18. A method of enhancing immune responses to an infectious disease in a subject in need thereof, comprising administering to the subject a therapeutically effective amount of the recombinant polypeptide of claim 1.


SEQUENCE LISTING (22 sequences)

SEQ ID NO: 1 - protein, 169 aa, synthetic construct
MYSMQLASCVTLTLVLLVNSAPTSSPTSSPTSSSTAEAQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 2 - protein, 215 aa, synthetic construct
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 3 - protein, 160 aa, synthetic construct
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 4 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 191 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETS

SEQ ID NO: 6 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 7 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 576 aa, synthetic construct
MYSMQLASCVTLTLVLLVNSAPTSSPTSSPTSSSTAEAQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGSGGGSGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGGGSGGGSGGGSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSGGHHHHHH

SEQ ID NO: 9 - protein, 536 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSGGGSGGGSGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGHHHHHH

SEQ ID NO: 10 - DNA, 1740 bp, synthetic construct
  source 1..1740: mol_type = other DNA
atgtacagcatgcagctcgcatcctgtgtcacactgacacttgtgctccttgtcaacagcgcacccacttcaagccccacttcaagccccacttcaagctctacagcggaagcacagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggtggaggttctggtggaggttctggtggaggttctgaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataagggtggaggttctggtggaggttctggtggaggttctagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagcggtggacatcaccatcaccatcacgggcccgggtaa

SEQ ID NO: 11 - DNA, 60 bp, synthetic construct
  source 1..60: mol_type = other DNA
atgtacagcatgcagctcgcatcctgtgtcacactgacacttgtgctccttgtcaacagc

SEQ ID NO: 12 - DNA, 447 bp, synthetic construct
  source 1..447: mol_type = other DNA
gcacccacttcaagccccacttcaagccccacttcaagctctacagcggaagcacagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 13 - DNA, 36 bp, synthetic construct
  source 1..36: mol_type = other DNA
ggtggaggttctggtggaggttctggtggaggttct

SEQ ID NO: 14 - skipped

SEQ ID NO: 15 - DNA, 645 bp, synthetic construct
  source 1..645: mol_type = other DNA
gaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataag

SEQ ID NO: 16 - DNA, 507 bp, synthetic construct
  source 1..507: mol_type = other DNA
agcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagcggtggacatcaccatcaccatcacggg

SEQ ID NO: 17 - DNA, 1584 bp, synthetic construct
  source 1..1584: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaactggtggaggttctggtggaggttctggtggaggttctgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttccggtggaggttctggtggaggttctggtggaggttctagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 18 - DNA, 60 bp, synthetic construct
  source 1..60: mol_type = other DNA
atgtacaggatgcaactcctgtcttgcattgcactaagtcttgcacttgtcacaaacagt

SEQ ID NO: 19 - DNA, 399 bp, synthetic construct
  source 1..399: mol_type = other DNA
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaact

SEQ ID NO: 20 - DNA, 573 bp, synthetic construct
  source 1..573: mol_type = other DNA
gagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcc

SEQ ID NO: 21 - DNA, 480 bp, synthetic construct
  source 1..480: mol_type = other DNA
agcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 22 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI